(19) United States
(12) Patent Application Publication
(10) Pub. No.: US 2014/0134268 A1
(43) Pub. Date: May 15, 2014

(54) HOMEOPATHIC COMPOSITIONS FOR TREATING BACK, SCIATIC, OR LUMBAR PAIN

(71) Applicant: Jean-Pierre Mageren, Seneffe (BE)

(72) Inventor: Jean-Pierre Mageren, Seneffe (BE)

(21) Appl. No.: 14/127,909

(22) PCT Filed: Jun. 13, 2012
(86) PCT No.: PCT/EP2012/061160

§ 371 Date: Dec. 19, 2013

(30) Foreign Application Priority Data
Jun. 23, 2011 (BE) 2011/0379

(51) Int. Cl. A61K 36/71; A61K 36/185; A61K 36/56; A61K 33/34

(52) U.S. Cl. CPC A61K 36/71; A61K 33/34; A61K 36/185; A61K 36/56
USPC 424/630

(57) ABSTRACT

The invention relates to a homeopathic composition for effectively treating and preventing back, sciatic, or lumbar pain. The composition includes Cuprum metallicum, Staphysagria, Ribes nigrum, and preferably includes Spigelia.

### TECHNICAL BACKGROUND OF THE INVENTION

The invention relates to a homeopathic composition intended to treat chronic back and lower limb pain. The invention particularly treats lumbar pain, such as lower back pain, lumbago, lumbar herniated disk and sciatica. The invention relates more particularly to the treatment of sciatica.

A report issued by ANAES (French National health accreditation and evaluation agency) relating to chronic lower back pain (Diagnostic, prise en charge et suivi des maladies atteints de lombalgie, December 2000) highlights how debilitating back pain can be and the potential consequences in everyday life. The guidelines in respect of the treatment methods used may also be applied to lumbar pain in general.

This report details the various allopathic treatments used and classified according to the intensity of the patient's pain. This report essentially relates to the use of analgesics.

Grade I analgesics such as paracetamol may be used occasionally to relieve pain; however, the use thereof involves some risks, particularly in the case of long-term use (notably a risk of ulcers) and no studies have demonstrated the efficacy of paracetamol in this indication.

Examples of grade I analgesics (non-opioid analgesics): Non-steroidal anti-inflammatory drugs, Acetylsalicylic acid, Paracetamol.

Grade II analgesics (weak opioids) such as morphine derivatives (codeine, dextropropoxyphene, etc.) and grade III analgesics (strong opioids only available on prescription in exceptional cases) help relieve pain, but the use thereof involve numerous adverse effects.

Furthermore, the duration of the treatment should be subject to a time limit.

Examples of grade II analgesics (weak opioid analgesics): Codeine, Nalbuphine, Dextroproxyphene, Tramadol, Oxycodone, Buprenorphine.

Examples of grade III analgesics (strong opioid analgesics): Morphine, Fentanyl, Hydromorphone.

The ANAES report also details the complementary use of:

Muscle relaxants (tetrazepam): These are used in cases of excessive and long-term contracture. However, the effects thereof are uncertain.

Tricyclic antidepressants: The effects thereof remain moderate.

The use of corticosteroids is not recommended according to this report.

Therefore, there is a need to treat back pain with an alternative method not involving the drawbacks or risks associated with occasional, or even long-term, use of analgesics, particularly risks of dependence and other undesirable side-effects.

Homeopathy consists of the use of one or a plurality of active agents or “stocks” used in the form of a homeopathic composition. The active agents are generally in the form of a mother liquor or mother tincture which is subsequently diluted in water or alcohol. There are a number of dilution methods in homeopathy, the most common being decimal (DH or X) or centesimal (CH) Hahnemannian dilutions or Korsakovian dilutions (K).

For example, a 6 DH solution is equivalent to 6 successive dilutions in a proportion of 1 to 9 of a mother tincture, generally in a mixture of water and alcohol.

After each dilution step, the mixture is shaken for around one hundred succussions. All these steps are defined as representing the dynamization of the solution. Centesimal dilutions (proportion of 1 to 99 for each dilution) are also found and denoted by C, along with “millennial” dilutions denoted by M (1 to 999). The techniques used are well known and are, for example, described in the “Guide du préparateur en pharmacie” (3rd edition, B. Charpentier et al., Ed. Masson).

As a general rule, low dilutions (4 or 5 CH) are recommended for treating acute illnesses whereas high dilutions (9 CH and over) are recommended for treating chronic illnesses.

The dose frequency depends on the patient's condition. In cases of acute illnesses, the doses should be frequent, whereas they will tend to be spaced out in cases of chronic illnesses.

The term “homeopathic complexes” denotes associations of homeopathic treatments intended to treat a particular problem.

The term homeopathic stock or active agent denotes the product, substance or initial composition for obtaining a homeopathic medicinal product.

The term mother tincture denotes the initial solution used for the dilutions for obtaining a homeopathic medicinal product. Generally, if the active agent is of plant or animal origin, the mother tincture serves as the stock.

Homeopathic medicinal products are generally presented in the form of vials, suppositories, drop formulas, eye washes, granules, triturations and ointments. According to the invention, it is more advantageous to use granules since the latter are suitable for easy dosage, on one hand, and highly convenient use during the day, on the other.

The term treatment infers that the invention is suitable for obtaining a curative and prophylactic action in respect of pain, particularly back and lower limb pain, such as sciatica.

The term lumbar pain denotes pain originating in the lower back. It may consist of lower back pain, lumbago, lumbar herniated disks and sciatica.

Pain is defined as chronic when it persists beyond the usual time for the presumed initial cause. Chronic pain is generally defined as being an “unpleasant sensory and emotional experience, associated with an existing or potential tissue lesion, or described in terms suggesting such a lesion” (“Douleur chronique: reconna\textcircled{i}tre le syndrome douloureux chronique, l′évaluer et orienter le patient”, French National Authority for Health, 2008, www.has-sante.fr).
Pain is what the subject feels, whether the cause thereof is identified or not.
The chronic pain included in these guidelines is a multidimensional syndrome expressed by the patient affected. Chronic pain is present, regardless of the topography and intensity thereof, when the pain has a plurality of the following features:
   persistence or recurrence, lasting for longer than the usual time for the presumed initial cause, particularly if the pain progresses for over 3 months;
   insufficient response to treatment;
   significant and progressive pain-induced degradation of the patient's functional and relational capacities in his/her day-to-day activities, at home and at school or work.

These back pains may have various triggering factors and the source thereof may be multiple: a bone, joint, disk, ligament and/or muscle problem.

Sciatica is caused by lesions in the spinal column and the intervertebral disks forming same. Intervertebral disks are subject to ageing, decreasing the elasticity thereof and weakening them accordingly. This may result in compression of the sciatic nerve giving rise to sharp pain in the thighs and may spread along the leg, to the calf, or even to the foot.

Lower back or back pain is generally not serious and the pain is spontaneously resolved after some time. In some cases, the back pain may be chronic, i.e. when it persists for more than 3 months, despite treatments.

Herniated disks occur when the disk is damaged to the point of continuously pressing on the nerve, causing sharp, intense pain.

The homeopathic treatment of back pain and sciatica is known, in particular the publication “Applied Homeopathy” (R. Jacobs and M. E. Pinkerson. Homeopathy Press, Santa Monica, Calif., 1983) gives an example of a homeopathic composition (Belladonna, Spigelia, Mag. phos. and Cimifuga wherein the dilution factor is 6×) for treating sciatica.

The publication “Vademecum de la prescription en homéopathie” (A. Horvilleur, Ed. Masson, ISBN 2-294-08111-0) also describes a homeopathic treatment for lower back pain; granules comprising Bryonia Alba 5 CH, Kalium Carbonicum 5 CH, Nux Vomica 5 CH, taken 3 times daily until the patient's condition improves.

This document also describes various homeopathic compounds frequently prescribed in the treatment of lumbar pain, in particular, Aesculus Hippocastanum, Berberis Vulgaris, Bryonia Alba, Colocynthis, Dulcamara, Kalium Carbonicum, Natrum Muriaticum, Nux Vomica, Rhus Toxicodendron, Sepia Officinalis, Actaea Recemosa, Calcarea Fluorica, Calcarea Phosphorica, Causticum, Helonias Dioica, Hypericum Perforatum, Lycopodium Clavatum, Phosphorus, Pulsatilla, Ruta Graveolens, Silicea, Sulfur.

Some stocks (Nux Vomica, Ruta Graveolens, etc.) are also prescribed for treating sciatica.

The publication “Traité d′homéopathie” (C. Gaucher, J-M Chabanne, Ed. Masson, ISBN 2-294-00703-4) describes numerous homeopathic compounds for treating back pain, lower back pain and sciatica. The document mentions, for example, the use of Medorrhinum and Dioscorea for treating sciatica. The document also mentions Radium Bromatum, Calcarea Carbonica, Calcarea Phosphorica, Natrum Muriaticum, Mandragora, Ferrum Metallicum, Senecio as effective compounds for treating lower back pain and lumbar pain.

Several publications mentioning numerous stocks are “Kent's Homeopathic Repertory” (MMI Ed. Masson, 2001, ISBN: 2-84650-002-9, ISSN: 1292-4873) contains for example, a non-exhaustive list of active agents with an action in the lumbar region. These include: Acon., Aesc,. Aeth., Agar., All.-c., All.-s., Aloe, Alum., Ambr., Am.-c., Am.-m, Anag., Ang., Ant-Ox., Apoc., Arg-m, Arg-n, Arn., Ars., Arund., Asaf., Aur., Aur.-m., Bar.-c., Bell., Berb., Bor., Brom., Bry., Bufo, Calad., Calc., Calc.-p., Camph., Canth., Caps., Carb.-ac., Carb.-an., Carb.-s., Carb.-v., Caust., Cham., Chel., Chin., Chin.-s., Cic., Cimic., Cimx., Cina., Cinnb., Clem., Cob., Cocc., Coc.-c., Colch., Coloc., Con., Conv., Cop., Corn, Cor-r., Cupr.-ar., Cur., Cycl., Dig., Dios., Dulc., Elaps., Eup.-per., Fago., Ferr., Ferr.-ar., Ferr.-i, Ferr.-p., Fl.-ac., Gamb., Graph., Grat., Ham., Hell., Hep., Hura., Hydrc., Hyos., Ign., Indq., Iris, Jarr., Nux-v, Kali.-br., Kali.-c., Kali.-i., Kali.-n., Kreos., Lac.-c., Lach., Lact., Led., Lept., Lil-t, Lob., Lyc., lyss., Mag.-c., Mab.-m., Mag.-s., Med., Meli., Meny., Merc., Merc.-Oci., Naja, Nat.-a., Nat.-c., Nat-m., Nat.-s., nicc., Nux-m., Nux-v., Osm., Ox-Ac., Ph.-ac., Phel., Phos., Phys., Pic-ac., Plan., Plat., Plb., Prun., Ptel., Psor., Puls., Ran.-s., Rhod., Rhus-t., Ruta, Sabad, Sabin, Sang., Sep., Sil., Spong., Staph., Stront., Stry., Sul.-ac., Sul.-i., Sulph., Tab., Thuj., Vib., Zinc.

Kent's Homeopathic Repertory also lists a number of ingredients suitable for treating sciatica effectively. These ingredients include: Agar., Am.-m., Anan., arg.-n., Arn., Ars., Asar., Bar-c., Bapt., Bell., Berb., Bry., Calc., Caps., Carb.-s., Card.-m., Caust., Cham., Chel., Chin.-s., Cimic., Cocc., Coc.-c., Coff., Coloc., Dios., Dros., Elat., Eup.-pur., Ferr., Gels., Gnaph., Guai., Hyper., Ign., Indg., Iris, Kali.-bi., Kali.-br., Kali.-c., Kali.-i., Kali.-p., Lac.-c., Lach., Led., Lyc., Lyss., Mag.-p., Merc., Menv., Meny., Mez., Nat.-a., Nat.-m., Nat.-s., Nit.-ac., Nux.-m., Nux.-v., Ol.-j., Pall., Petr., Ph.-ac., Phos., Phyt., Plan., Plat., Plb., Podo., Psor., Puls., Ran.-b., Rhod., Rhus.-t., Ruta, Sal.-ac., Sep., Sil., Staph., Still., Stram., Sulph., Syph., Tell., Ter., thuj., Valer., Verat., Xan., Zinc.

Moreover, numerous stocks are used in homeopathy for the anti-inflammatory effects thereof. Mention may be made, for example, of Aconitum, Aconitum nap, Arnica, Bryonia, Eupatorium perf, Hypericum Ruta Symphytum (Homéopathie pratique, Dr. C. Binet, Ed. Dangles, p. 30, 93, 159, 176) Apis, Harpagophytum, Salix, Harpagophytum, Hamamelis, Fraxinus, Tilia, Calluna. These ingredients are suitable for relieving some types of inflammatory pain. However, the efficacy thereof is not suitable for obtaining a specific action for the treatment and/or prevention of lumbar pain.

The application US 2005/0100513 relates to a homeopathic composition comprising Staphysagria and/or Spigelia. This composition is intended to treat nicotine addiction and has not been described by the inventors thereof as having an effect for the treatment of back pain. For this reason, this application relates to a field far removed from that of the invention.

The application WO 2010/57295 describes a homeopathic composition comprising Cuprum and Spigelia for treating neuropathic pain and also relates to a field far removed from that of the invention.

The patents EP 1 236 466 and U.S. Pat. No. 5,683,712 mention a homeopathic composition comprising Spigelia described in “Applied Homeopathy” (R. Jacobs and M. E. Pinkerson, Homeopathy Press, Santa Monica, Calif., 1983). This composition is suitable for treating sciatic pain. However, the composition described consists of a specific combination of four active ingredients (Belladonna 6×, Spigelia 6×, Mag. Phos. 6× and Cimifuga 6×) which would not be suitable for obtaining a preventive effect, this composition not comprising a homeopathic anti-inflammatory agent suitable for inducing corticoadrenal stimulation.

“More Ultimate Healing”, (2007 Jan. 1), Bottom Line Books describes the homeopathic use of Cuprum Metallicum for treating back pain. “The Complete Homeopathic Resource for Common Illnesses”, (2006 Jan. 1), Denis Chermin, North Atlantic Books, Berkley Calif., describes that of Staphysagria for treating lower back pain. However, these compositions are not effective for treating chronic lumbar pain and are not suitable for preventing the onset of back pain.

“Secrets of Native American Herbal Medicine”, (2001 Jan. 1), Avery, Penguin Putnam, describes the uses of Ribes Nigrum for relieving back pain; however, no homeopathic treatment is described.

One of the aims of the invention relates to a homeopathic composition suitable for obtaining a prophylactic and preventive action for treating lumbar pain such as lower back pain, lumbago, herniated disks and sciatica, particularly chronic pain.

A further aim of the invention relates to a homeopathic composition which is particularly effective for relieving lumbar pain, notably chronic lumbar pain.

A further aim of the invention relates to a homeopathic composition which is particularly effective for relieving sciatica.

A further aim of the invention relates to a homeopathic composition used preventatively to prevent lumbar pain.

A further aim of the invention relates to a homeopathic composition used preventatively to prevent sciatica.

### DESCRIPTION OF THE FIGURES

FIG. 1 is a graph illustrating the progression of chronic lumbar pain over a 60-day period on 7 subjects treated with homeopathy.

FIG. 2 illustrates the progression of pain over a 24-hour period on 9 subjects treated with homeopathy.

### DETAILED DESCRIPTION OF THE INVENTION

To the inventor's knowledge, there are no homeopathic compositions comprising Cuprum, Staphysagria and Ribes Nigrum suitable for obtaining a preventive and curative action on back and/or lower limb pain such as lower back pain and herniated disks, notably chronic pain.

The characteristics of the stocks used are referenced exhaustively for example by “The Homoeopathic Pharmacopoeia of the United States” (HPUS) and by other databases. The publication “Homéopathie pratique, Dr. C. Binet, Ed. Dangles” describes:

CUPRUM (“Homéopathie pratique” p. 70)

This consists of copper in the metallic form thereof. This substance is used in homeopathy for treating various conditions including muscle cramps and contractions such as spasms.

RIBES NIGRUM (“Homéopathie pratique” p. 156)

Blackcurrant, Grossulariaceae, which is known for the diuretic and detoxifying properties thereof. Ribes Nigrum is above all used for allergies, the use thereof notably enabling corticoadrenal stimulation inducing anti-inflammatory hormone secretion.

SPIGELIA (“Homéopathie pratique” p. 170)

Spigelia Anthelmia (anthelmintic) or Demerara Pink-root is a plant known and used for treating heart palpitations, angina pectoris, endocarditis and valvular disease.

Spigelia is also known as being particularly effective for treating rheumatoid arthritis associated scleritis.

A further use of Spigelia relates to the treatment of any disorders associated with the presence of intestinal parasites.

STAPHYSAGRIA (“Homéopathie pratique” p. 172)

Also known as lice-bane, Staphysagria is a Ranunculaceae used in homeopathy for treating urinary tract and genital itching with alternating skin disorders and rheumatism.

The homeopathic compositions used to date for treating back pain have proved to be relatively ineffective. However, the inventor observed that the combination of Cuprum, Ribes Nigrum and Staphysagria in a homeopathic composition is suitable for enhancing the treatment of these pains significantly.

The inventor formulated and developed a particularly advantageous homeopathic composition since it is suitable for obtaining a preventive effect in addition to a curative effect and does not involve the side-effects expected when using the active agents separately. Moreover, said composition exhibits a remarkable effect for relieving chronic back pain.

The use of Staphysagria is known in homeopathy as a muscle relaxant and may also be used for relieving muscle pain. Staphysagria is also used in numerous homeopathic compositions intended to relieve back pain.

Staphysagria is suitable for relieving and soothing pain, and would be suitable for obtaining, in cases of back pain, a localized action on the lower limbs and on the back in the lumbar region.

Ribes Nigrum is generally used in homeopathic compositions to stimulate the release of cortisone produced by the body, thus obtaining an anti-inflammatory effect, unlike that obtained using Staphysagria. A more comprehensive description of the properties of Ribes Nigrum may be obtained in the publication “Homéopathie pratique” (Dr. C. Binet, Ed. Dangles, p. 156). Following the use of Ribes Nigrum, it is also frequent to observe the onset of muscle cramps.

The inventor observed that adding Cuprum Metallicum in no way affects the efficacy of Ribes Nigrum and Staphysagria for treating back pain and would be suitable, furthermore, for eliminating or substantially reducing the onset of muscle cramps and any other form of contractures.

Moreover, the use of this homeopathic composition is suitable for obtaining a particularly effective preventive and prophylactic effect on the onset of lumbar pain. Without being bound to any scientific theory, it would appear that the prophylactic effect is associated with the use of Ribes Nigrum which is suitable for releasing cortisone in the body and would thus protect the body from any pain.

Furthermore, adding Spigelia to the composition offers a further advantage. Spigelia is an active agent used in homeopathy. When a subject suddenly discontinues an allopathic treatment, in particular pain relief or anti-inflammatory medication, a withdrawal syndrome frequently occurs; for example, there may be dependence on substances such as codeine. The use of Spigelia tends to offset and relieve this withdrawal syndrome and enable withdrawal. It is thus advantageous to use Spigelia at the start of treatment particularly for patients having undergone an allopathic treatment.

A homeopathic composition as developed comprising Cuprum Metallicum, Staphysagria and Ribes Nigrum is thus particularly effective in preventing, relieving and treating back pain.

Preferably, the homeopathic composition is used in a dilution between 5 and 7 CH (centesimal Hahnemannian). The homeopathic composition advantageously comprises Cuprum Metallicum and Ribes Nigrum in a 5 CH dilution and Staphysagria in a 7 CH dilution. More advantageously, the homeopathic composition also contains Spigelia in a dilution between 5 and 7 CH and preferably of 5 CH.

The preventive aspect is obtained by taking 2 to 3 granules at least one hour, preferably between 1 and 8 hours, before physical exertion liable to give rise to back pain, making it possible to prevent the onset of lumbar and/or sciatic pain.

The lumbar pain treatment is preferably obtained by taking 2 granules 3 times daily. This treatment may be occasional, i.e. after the onset of lumbar pain or following exertion. The treatment may also be extended, for example in cases of chronic pain. In this case, the preferential treatment consists of taking 2 granules 3 times daily for a period of 3 to 6 months. Following this treatment, an improvement in the patients' condition is observed. Full patient recovery may also be observed if the treatment is continued for further period of 3 to 6 months.

### Examples

### Pain Measurement

The perceived pain is rated by the patient him/herself on a rating scale in the form of a simple verbal scale (SVS) including 10 graduations, each corresponding to an adjective describing pain according to the intensity thereof: none (0), mild (2), discomforting (4), severe (6), unbearable (8), maximum imaginable (10).

Besides the patient's verbal statement, the physician's observation is taken into consideration.

Examples 1-6 correspond to patients using a homeopathic composition comprising: Cuprum Metallicum (Cupr. Metall.) 5 CH, Staphysagria (Staph.) 7 CH, Ribes Nigrum 5 CH, Spigelia 5 CH.

Examples 7 and 8 correspond to patients using a homeopathic composition comprising: Cuprum Metallicum 5 CH, Staphysagria 7 CH and Ribes Nigrum 5 CH.

The homeopathic composition may be obtained by diluting a mother tincture in a suitable solvent, preferably water and/or alcohol. Each of these dilution steps is followed by a succussion step which consists of shaking the solution vigorously at least 100 times. The mixture is then diffused, on inert granules.

The stocks used by the present invention are marketed in diluted form by BOIRON under the references SPIGELIA (HAB), STAPHYSAGRIA (PHFR), RIBES NIGRUM GEMMAE MG (HAB), CUPRUM METALLICUM 5 CH.

The inert granules were supplied by Debofar under the reference PF125 (85/15) 4 mm.

According to the preferred embodiment, the mixture comprises 1 ml of 70 deg Ribes Nigrum 5 CH, 1 ml of 70 deg Staphysagria 7 CH, 1 ml of 70 deg Spigelia 5 CH and 1 ml of 70 deg Cuprum Metallicum 5 CH. The mixture is then diffused, on 60 g of previously calibrated neutral sucrose or glucose granules.

TABLE 1
Results obtained on several patients:

| | | Treatment | Effects/ |
|---|---|---|---|
| Case | Treatment | period | observations |
| 1. Sciatica | 2 granules 3 | 3 months | Complete |
| | times daily | | disappearance |
| | | | of pain |
| 2. Stiffness | 2 granules 3 | 1 month | Helped reduce |
| and curved | times daily | | pain relief and |
| posture | In addition to | | anti- |
| | pain relief and | | inflammatory |
| | anti- | | medication |
| | inflammatory | | Straightening |
| | medication | | of posture |
| Follow-up of | | 3 months | Full recovery |
| case 2. | | | |
| 3. Sciatica | 2 granules 3 | 1 month | Full recovery |
| | times daily | | |
| | In addition to | | |
| | pain relief and | | |
| | anti- | | |
| | inflammatory | | |
| | medication | | |
| 4. Chronic back | 3 granules 3 | 1 to 3 months | Helped reduce |
| pain | times daily | | pain relief and |
| | | | anti- |
| | | | inflammatory |
| | | | medication |
| 5. Sciatica | 3 granules 3 | 1 month | Helped reduce |
| | times daily | | pain relief and |
| | | | anti- |
| | | | inflammatory |
| | | | medication |
| Follow-up of | 3 granules 3 | 6 months | Discontinuation |
| case 5. | times daily | | of pain relief |
| | | | and anti- |
| | | | inflammatory |
| | | | medication. |
| 6. Sciatica | 2 granules 3 | 15 days | Full recovery |
| | times daily | | |
| 7. Pain | 3 granules | A few hours | Effective |
| following | immediately and | | disappearance |
| physical | then every 2 | | of pain after a |
| exertion | hours until | | few hours |
| | pain clears | | |
| 8. Preventive | 2 to 3 granules | | No pain for the |
| effect | taken 1 to 8 | | 8 hours |
| | hours before | | following |
| | exertion | | administration |
| | | | of granules |

Case 1: Relates to a 50 year old man, injured and bedridden following a car crash and suffering from sciatica. An improvement in his condition was observed after 10 days of treatment; pain had fully cleared after 3 months.

Case 2: Relates to a 50 year old farmer suffering from chronic lower back pain having a curved posture and taking 1 to 3 anti-inflammatory tablets (Arcoxia) weekly. The treatment consisted of taking 2 granules 3 times daily for one month, in addition to anti-inflammatory medication. Subsequently, the pain relief and anti-inflammatory medication was progressively reduced before full discontinuation. After 1 month, straightening of the posture was observed. After 3 months, complete straightening was observed.

Case 3: Relates to a 53 year old man suffering from sciatica induced by his occupation (sports gym security guard) which required him to remaining standing. The treatment consisted of taking 2 granules 3 times daily, in addition to anti-inflammatory medication. After one month of treatment, it was possible to completely discontinue the pain relief and anti-inflammatory medication and a complete disappearance of the pain was observed.

Case 4: Relates to a 25 year old young woman suffering from chronic back pain. She replaced the pain relief and anti-inflammatory medication (Dafalgan) by 3 granules of the homeopathic composition according to the application.

Case 5: Relates to a 55 year old man suffering from sciatica. Taking 3 granules 3 times daily for 1 month helped reduce the pain relief and anti-inflammatory medication (Dafalgan Codeine and Feldene) and, by extending the administration of 3 granules 3 times daily for 6 months, made it possible to discontinue the pain relief and anti-inflammatory medication completely.

Case 6: Relates to a woman approximately fifty years of age, having sciatica following a physical attack. Taking 2 granules 3 times daily for 15 days helped clear the pain.

Case 7: Relates to 20-25 year old young laborers working in construction and suffering from back pain following physical exertion. Taking 3 granules according to the invention helped relieve and clear the pain.

Case 8: Relates to a preventive treatment on the onset of back pain on two subjects of 61 and 23 years of age working on a construction site. Taking 3 granules according to the invention morning before work helped prevent the onset of back pain over the 3-year period corresponding to the duration of the project.

Chronic Lumbar Pain

The study was conducted over a period of two years. A group of 7 subjects, male and female, from 40 to 65 years of age and suffering from chronic lumber pain were selected after checking the following conditions:
checking by means of conventional medical examinations particularly blood tests and X-rays that lumbar pain was actually involved and not cases of rheumatism, arthritis or arthrosis.
Checking that the lumbar pain dated back over 3 months previously and that anti-inflammatory medication had remained ineffective.
Checking medical treatments to exclude any patient not suffering only from chronic lumbar pain but also suffering from rheumatism, arthritis or arthrosis in association with chronic lumbar pain.

The patients' initial condition was rated according to the SVS scale method, consisting of the patient rating themselves on a scale ranging from 0 to 10, where 0 denotes no pain and 10 denotes intolerable pain. The rating included the following questions:
When you are experiencing severe pain, are you capable of any physical activity?
When you are experiencing severe pain, are you capable of any intellectual activity?
When you get up each day, do you feel able to work?

To evaluate the efficacy of each active agent, a control group made up of 3 subjects received 2 homeopathic granules three times daily a quarter of an hour before mealtimes for 1 month. Each of these subjects was only treated with granules comprising a complex consisting either of Cuprum Metallicum 5CH, or Staphysagria 7CH or Ribes Nigrum 5CH.

The 4 other subjects taking part in evaluating the complex according to the invention received a homeopathic medicinal product comprising Cuprum Metallicum 5 CH, Staphysagria 7 CH and Ribes Nigrum 5 CH. The dosage was identical to that of the control group, i.e. taking 2 homeopathic granules three times daily for 1 month.

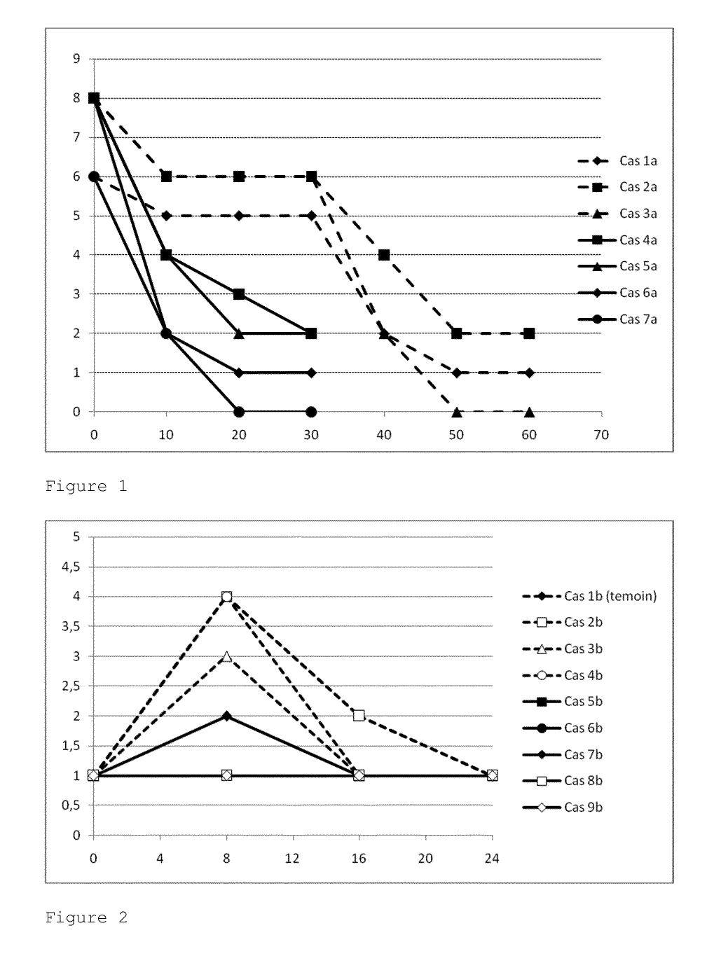

Perceived pain was rated by each patient on rising at 10, 20 and 30 days of treatment. The results of these tests are summarized in Table 2 hereinafter and illustrated by the graph in FIG. 1.

TABLE 2
Pain measurement based on SVS scale (0-10).

| Case | 1a | 2a | 3a | 4a | 5a | 6a | 7a |
|---|---|---|---|---|---|---|---|
| Active | Cupr. Metall. | Staph. 7 | Ribes | Cupr. Metall. 5 | Cupr. Metall. 5 | Cupr. Metall. 5 | Cupr. Metall. 5 |
| agents | 5 CH | CH | Nigrum | CH, Staph. 7 | CH, Staph. 7 | CH, Staph. 7 | CH, Staph. 7 |
| | | | 5 CH | CH and Ribes | CH and Ribes | CH and Ribes | CH and Ribes |
| | | | | Nigrum 5 CH | Nigrum 5 CH | Nigrum 5 CH | Nigrum 5 CH |
| Before | 6 | 8 | 8 | 8 | 8 | 8 | 6 |
| taking | | | | | | | |
| medication | | | | | | | |
| After 10 | 5 | 6 | 6 | 4 | 4 | 2 | 2 |
| days | | | | | | | |
| After 20 | 5 | 6 | 6 | 3 | 2 | 1 | 0 |
| days | | | | | | | |
| After 30 | 5 | 6 | 6 | 2 | 2 | 1 | 0 |
| days | | | | | | | |
| Active | Cupr. Metall. 5 | Cupr. Metall. 5 | Cupr. Metall. 5 | | | | |
| agents | CH, Staph. 7 | CH, Staph. 7 | CH, Staph. 7 | | | | |
| | Ch and Rise | Ch and Rise | Ch and Rise | | | | |
| | Nigrum 5 CH | Nigrum 5 CH | Nigrum 5 CH | | | | |
| After 40 | 2 | 4 | 2 | | | | |
| days | | | | | | | |
| After 50 | 1 | 2 | 0 | | | | |
| days | | | | | | | |
| After 60 | 1 | 2 | 0 | | | | |
| days | | | | | | | |

Over a 1-month period, it is observed that, the subjects in the control group (cases 1a, 2a and 3a) following a treatment with one of the active agents exhibit little or no improvement in terms of the perceived intensity of their pain (FIG. 1).

The subjects (cases 4a, 5a, 6a, 7a) receiving a treatment according to the invention experienced a significant decrease in the perceived pain intensity (FIG. 1).

It was thus confirmed that taking a medicinal product consisting of a complex according to the invention is suitable for obtaining rapid chronic pain relief in patients with effects observed from the start of treatment.

In a subsequent step, the control group receiving the granules comprising one of the active agents was treated with a homeopathic composition according to the invention (cases 1a-3a). The treatment consists of taking 2 granules three times daily for 1 month.

It was confirmed that taking a medicinal product consisting of a complex according to the invention is suitable for obtaining chronic pain relief in patients with effects observed from the start of administration of the medicinal product comprising the active agent.

Treating these patients with a homeopathic medicinal product comprising a complex consisting of Cuprum Metallicum 5 CH, Staphysagria 7 CH and Ribes Nigrum 5 CH, helped reduce chronic pain significantly.

### Evaluation of Medicinal Product in Respect of Preventing Lumbar Pain.

A study was conducted to measure the preventive effects of the homeopathic medicinal product on the onset of back pain.

This study was conducted over a period of two years and was performed on a group of 9 subjects from 18 to 65 years of age working as a manual worker or laborer in construction, warehouse operative, delivery driver, auto mechanic, each of these subjects suffering from lumbar pain.

The patients suffering from lumbar pain induced by exertion were selected after checking the following conditions:
Checking medical treatments to exclude any patient not suffering only from chronic lumbar pain but also suffering from rheumatism, arthritis or arthrosis.
Checking whether the lumbar pain is associated with occupational exertion following a 24-hour cycle including 3×8-hour periods.
Exclusion of cases of rheumatism, arthritis or arthrosis by means of conventional medical examinations, notably by means of blood testing or X-rays.

The patients' initial condition was rated according to the SVS scale method, on a graduated pain scale from 0 to 10, where 0 denotes no pain and 10 denotes the maximum pain imaginable. The rating included the following questions:
“On rising, rate the pain intensity on the graduated SVS scale from 0 to 10”
“After a day's work, rate the pain intensity on the graduated SVS scale from 0 to 10”
“When going to bed, rate the pain intensity on the graduated SVS scale from 0 to 10?”

Every day, the patient reports the lumbar pain measurement on the SVS scale after rising; after a day's work and before going to bed. The mean values of these measurements are reported in FIG. 2.

Cases 1b-9b were treated preventatively by administering 2 homeopathic granules once a day, for one week, 1 hour before starting work.

Cases 1b to 4b represent a group of control subjects respectively using granules not comprising active agent, granules consisting of Cuprum Metallicum 5 CH, granules consisting of Staphysagria 7 CH, granules consisting of Ribes Nigrum 5 CH.

Cases 5b to 9b received granules of homeopathic medicinal product according to the invention and consisting of Cuprum Metallicum 5 CH, Staphysagria 7 CH and Ribes Nigrum 5 CH.

The pain intensity was then recorded at different times of the day. The means of the results obtained over the week are reported in Table 3.

| | Case | | | | | | | | |
|---|---|---|---|---|---|---|---|---|---|
| | 1b | | | | | | | | |
| | (control) | 2b | 3b | 4b | 5b | 6b | 7b | 8b | 9b |
| Active | None | Cupr. Metall. | Staph. | Ribes | Cupr. Metall. | Cupr. Metall. | Cupr. Metall. | Cupr. Metall. | Cupr. Metall. |
| agents | | 5 CH | 7 CH | Nigrum | 5 CH, Staph. | 5 CH, Staph. | 5 CH, Staph. | 5 CH, Staph. | 5 CH, Staph. |
| | | | | 5 CH | 7 CH and Ribes | 7 CH and Ribes | 7 CH and Ribes | 7 CH and Ribes | 7 CH and Ribes |
| | | | | | Nigrum 5 CH | Nigrum 5 CH | Nigrum 5 CH | Nigrum 5 CH | Nigrum 5 CH |
| Pain intensity | 1 | 1 | 1 | 1 | 1 | 1 | 1 | 1 | 1 |
| on rising | | | | | | | | | |
| (mean value | | | | | | | | | |
| over one week) | | | | | | | | | |
| Pain intensity | 4 | 4 | 3 | 4 | 1 | 1 | 2 | 1 | 1 |
| 8 h after | | | | | | | | | |
| rising | | | | | | | | | |
| (mean value | | | | | | | | | |
| over one week) | | | | | | | | | |
| Pain intensity | 2 | 2 | 1 | 1 | 1 | 1 | 1 | 1 | 1 |
| 16 hours after | | | | | | | | | |
| rising | | | | | | | | | |
| (mean value | | | | | | | | | |
| over one week) | | | | | | | | | |
| Pain intensity | 1 | 1 | 1 | 1 | 1 | 1 | 1 | 1 | 1 |
| 24 hours after | | | | | | | | | |
| rising | | | | | | | | | |
| (mean value | | | | | | | | | |
| over one week) | | | | | | | | | |

Progression of Pain

FIG. 2 shows the progression of pain over a 24-hour period starting when the subject rises.

The day may be divided into three periods of approximately 8 hours after rising, equivalent to 8 hours of work activity, 8 hours of social life and 8 hours of rest and sleep.

The subject generally starts the working day with no lumbar pain. Indeed, on rising, the lumbar pain induced by physical exertion is zero or practically zero. In the course of the day, the lumbar pain increases progressively up to a level described as discomforting (cases 1b to 4b).

During the 8-hour period following the period of work activity, a progressive decrease in pain intensity is observed, tending to disappear completely in the third 8-hour period corresponding to the patient's period of rest and sleep.

During these tests, the inventor observed that using Cuprum Metallicum, Staphysagria or Ribes Nigrum separately was not suitable for obtaining a preventive effect on the onset of back pain, the pain perceived by cases 2b-4b during their period of activity being similar to that perceived by the control patient (case 1b).

The complex according to the invention consisting of Cuprum Metallicum, Staphysagria and Ribes Nigrum is thus suitable for obtaining a preventive effect for cases 5b to 9b, which is observed by a lack of increase in pain in the course of the day.

Key of figures
FIG. 1

| | |
|---|---|
| Cas | Case |

FIG. 2

| | |
|---|---|
| Cas | Case |
| (témoin) | (control) |

1. A Homeopathic composition comprising: Cuprum Metallicum, Staphysagria and Ribes Nigrum.

2. The homeopathic composition according to claim 1, further comprising Spigelia.

3. The homeopathic composition according to claim 1, wherein the dilution of each homeopathic stock is between 5 and 7 CH.

4. The homeopathic composition according to claim 3, wherein the dilution of the homeopathic stocks is 5 Ch for Cuprum Metallicum and Ribes Nigrum and 7 CH for Staphysagria.

5. The homeopathic composition according to claim 4, wherein the composition comprises Spigelia at a 5 CH dilution.

6. The homeopathic composition according to claim 1, for the application thereof as a medicinal product with a preventive effect on lumbar pain including lower back pain, lumbago, lumbar herniated disks and sciatica, wherein the medicinal product is used at least one hour before exertion liable to give rise to lumbar and/or sciatic pain.

7. The homeopathic composition according to claim 1, for the application thereof as a medicinal product for relieving pain including lower back pain, lumbago, lumbar herniated disks and sciatica, wherein the medicinal product is taken 2 to 3 times daily when said pain occurs.

8. The homeopathic composition according to claim 1, for the application thereof as a medicinal product for treating chronic lumbar pain including lower back pain, lumbago, lumbar herniated disks and sciatica, wherein the medicinal product is taken 2 to 3 times daily for an extended period of 3 to 6 months.

9. The homeopathic medicinal products comprising the homeopathic composition according to claim 1, wherein the medicinal product is packaged in one of the following forms: vials, suppositories, drop formulas, eye washes, granules, triturations, ointments.

10. The homeopathic composition according to claim 2, for the application thereof as a medicinal product with a preventive effect on lumbar pain including lower back pain, lumbago, lumbar herniated disks and sciatica, wherein the medicinal product is used at least one hour before exertion liable to give rise to lumbar and/or sciatic pain.

11. The homeopathic composition according to claim 2, for the application thereof as a medicinal product for relieving pain including lower back pain, lumbago, lumbar herniated disks and sciatica, wherein the medicinal product is taken 2 to 3 times daily when said pain occurs.

12. The homeopathic composition according to claim 2, for the application thereof as a medicinal product for treating chronic lumbar pain including lower back pain, lumbago, lumbar herniated disks and sciatica, wherein the medicinal product is taken 2 to 3 times daily for an extended period of 3 to 6 months.

13. The homeopathic medicinal products comprising the homeopathic composition according to claim 2, wherein the medicinal product is packaged in one of the following forms: vials, suppositories, drop formulas, eye washes, granules, triturations, ointments.
